(19) United States
(12) Patent Application Publication
(10) Pub. No.: US 2008/0082091 A1
(43) Pub. Date: Apr. 3, 2008

(54) Fiber optic tissue ablation

(76) Inventors: Vladimir Rubtsov, Los Angeles, CA (US); Hillel Laka, Beverly Hills, CA (US); Michael Levin, Los Angeles, CA (US); Minoo Akbarian, Los Angeles, CA (US); Igor Peshko, Mississauga (CA)

Correspondence Address: Lawrence S. Cohen, Suite 1220, 10960 Wilshire Blvd., Los Angeles, CA, 90024, US

(21) Appl. No.: 11/518,639

(22) Filed: Sep. 10, 2006

(51) Int. Cl. A61B 18/18

(52) U.S. Cl. 606/17

(57) ABSTRACT

A catheter tip for delivering laser light energy to create lesions extending to a depth of several millimeters in tissue includes an elongated, flexible housing. A reflector is oriented longitudinally in a channel the housing. A side emitting optical fiber diffuser, extending the length of the reflector, is held at a fixed separation from the reflector. The reflector and the side emitting diffuser are configured and spaced to provide a convergent beam directed through the tissue under treatment. Reflector curvature may be circular or elliptical in cross section, or other selected shape. The diffuser and reflector relative positioning being selected to place the beam focal point (or image) at a predetermined lateral distance from the reflector preferably not closer than the far wall of the tissue under treatment. Temperature probes are provided to monitor the temperature gradient through the tissue thickness. Optionally cooling and/or irrigation fluid are provided. Optionally, the fiber terminates at a retro-reflector.

### GOVERNMENT LICENSE RIGHTS

This invention was made with Government support under grant number IR43HL079734-01 from the National Institutes of Health. The Government has certain rights in the invention.

### BACKGROUND

1. Field of the Invention

The present invention relates to medical interventional applications of fiber optics and especially to delivery of intense infrared for heating tissues.

2. General Background and State of the Art

The condition known as atrial fibrillation (AF) is characterized by rapid and irregular activation of the atria which leads to the loss of normal sinus rhythm, and contributes significantly to cardiovascular morbidity and mortality. The preferred surgical treatment of AF, known as a “maze procedure,” involves cutting the heart tissue to produce a pattern of lesions which tend to block the propagation of the irregular electrical activity that maintains the fibrillation. Because the maze procedure is complex and prolonged and entails cardiopulmonary bypass, interventionists have pursued alternative means of creating lesions, most commonly by delivering radio frequency energy to the heart tissue through a catheter. Unfortunately, these procedures can damage the heart. Thus, alternatives to radio frequency energy have been explored. Among those alternatives is laser energy delivered through optical fibers, typically involving emission of laser radiation from the end of the fiber or from a quartz rod attached to the end.

However, end-emitting laser technologies have not met the needs of interventionists performing partial maze procedures. In order to block the pathways of electrical activity in AF, an interventionist needs to create long, continuous lesions in the heart tissue. Additionally, the lesions should be created quickly and without inflicting excessive damage on the anatomical structures of the heart. An end-emitting fiber is able generally to deliver laser energy to only one location at a time. Thus, there is a need to be able quickly to deliver laser radiation of consistent intensity to a long strip of tissue. To be permanent, the lesions should be approximately 4 millimeters in depth. Therefore, it is desirable to deliver laser energy to tissue over a depth range of 0 to 4 millimeters along the entire strip of tissue that is to be lesioned. Because tissue absorbs and scatters the laser radiation, an attempt to deliver sufficiently intense radiation to assure a lesion at a depth of 4 millimeters may have the undesired effect of delivering overly intense radiation at a lesser depth, charring or vaporizing the intervening tissue and creating coagulum which may adhere to the emitting apparatus and interfere with its operation. Thus, it is also desirable to be able to selectively deliver sufficiently intense radiation to tissue at depths approaching 4 millimeters without overheating tissue at a lesser depth.

In order to perform the procedure safely and quickly, the fiber optic apparatus conducting the laser energy should be carried on a catheter flexible enough to enable the interventionist efficiently to create the lesions on a complex curved surface of the heart. Additionally, the safety and consistency of the procedure can benefit from the ability to measure and control the temperature of tissues at various depths adjacent the site of the lesion during the procedure and to adjust the intensity or duration of the laser irradiation based on the measured temperatures. Thus, it would be helpful to utilize a catheter equipped with one or more temperature probes.

It is also important that the catheter follow the shape of the tissue to be treated and should be quickly applied, held firmly in place during the procedure and easily removed.

### SUMMARY

It is an object of the present invention to provide improved apparatus and methods for using laser quickly and efficiently to create elongated, lesions of precisely controlled depth and severity in vivo.

In accordance with these objects and with others which will be described and which will become apparent, an exemplary embodiment of tissue ablation apparatus in accordance with the present invention includes an elongate housing having a tissue contacting surface, defined as a tissue/catheter contact plane; a fiber optic waveguide operatively connected to a side emitting diffuser, the side emitting diffuser being disposed on the housing and an elongate reflector disposed on the housing in predetermined spatial relation to the tissue contacting surface and the side emitting diffuser in turn being in predetermined spatial relationship to the reflector. The reflector and the side emitting diffuser define at least one convergent beam at a predetermined location relative to the tissue contacting surface

In an exemplary embodiment of tissue ablation apparatus in accordance with the present invention the tissue contacting surface includes at least one suction orifice and the housing includes at least one suction channel in fluid communication with the orifice. Typically several suction orifices will be spaced along the length of the housing to ensure rapid self-attachment to the tissue.

In an exemplary embodiment the tissue contacting surface includes at least one temperature probe. Typically temperature thermocouples are spaced apart along the housing near the fiber and extend into the tissue to measure temperature gradient through the tissue thickness

In an exemplary embodiment, the housing includes at least one irrigation orifice located proximate the fiber optic waveguide and at least one irrigation channel in fluid communication with the irrigation orifice.

In an exemplary embodiment, at least one spacer projects from the tissue contacting surface defining the tissue/catheter contact plane.

In an exemplary embodiment, the side emitting diffuser emits energy substantially uniformly over its length.

In an exemplary embodiment, the reflector partially surrounds the side emitting diffuser and the side emitting diffuser being held at a predetermined lateral separation from the sireflector, the lateral separation being substantially constant over the length of the side emitting diffuser.

In an exemplary embodiment, the tissue/catheter contact plane defines a substantially planar area of contact at a substantially fixed lateral distance from the reflector, and wherein the convergent beam is defined at a predetermined location within a predetermined range of lateral distances from the reflector and consequently also from the diffuser, the range beginning at the area of contact and extending a predetermined distance beyond the area of contact.

In an exemplary embodiment, the side emitting diffuser, the reflector, and the tissue contacting surface have a length in a range between five and ten centimeters.

In an exemplary embodiment, the housing, the tissue/catheter contact plane the side emitting diffuser and the reflector are deformable between a straight configuration and a curved configuration and wherein a substantially fixed predetermined separation is maintained between the side emitting diffuser and the reflector, and between the reflector and the tissue/catheter contact plane, at the straight configuration, at the curved configuration, and at intermediate configurations.

In an exemplary embodiment, the side emitting diffuser is a length of fiber imprinted with a long period grating and wherein the side emitting diffuser emits energy substantially uniformly over its length. A preferred wavelength for the laser source is between 970 and 1070 nanometers, more preferably 970 to 980 nanometers.

In an exemplary embodiment, the side emitting diffuser terminates distally into a structure selected from the group including a corner cube retro-reflector, a right angle prism, and a multilayer dielectric mirror, among other structures that can provide retro-reflectivity.

Also in accordance with the present invention, an exemplary embodiment of tissue ablation apparatus includes an elongate, an elongate flexible housing having elements that provide surfaces to define a tissue/catheter contact plane; a plurality of suction orifices formed in portions of the housing, the suction orifices being spaced apart and opening at the tissue/catheter contact plane; at least one suction channel, located in the housing, in fluid communication with the suction orifices; a fiber optic flexible side emitting diffuser having energy emission substantially uniform over its length, the side emitting diffuser being disposed within the housing; and an elongate, flexible reflector disposed within the housing and the three structural elements, the surfaces defining the tissue/catheter contact plane. The reflector and the diffuser all being spatially located to provide a convergent beam configured to have a focal point in relation to a tissue under treatment such that the focal point is not closer than the distal wall of the tissue. The reflector extends substantially alongside the side emitting diffuser and partially surrounds the side emitting diffuser. The reflector and the side emitting diffuser define at least one convergent beam at a predetermined lateral distance from the side emitting diffuser. The predetermined lateral separations and the predetermined lateral distance are substantially unaffected by flexion of the housing, tissue contacting surface, side emitting diffuser, and reflector over a predetermined flexional range. The tissue contacting surface defines a substantially planar area of contact (the tissue/catheter contact plane) at a substantially fixed lateral distance from the side emitting diffuser. The convergent beam is defined at a predetermined location within a predetermined range of lateral distances from the side emitting diffuser, the range beginning at the area of contact and extending a predetermined distance beyond the area of contact.

In an exemplary embodiment, the convergent beam is defined at a location within a range between zero and four millimeters beyond the distal wall of the tissue, and is preferably not closer than the distal wall of the tissue.

In an exemplary embodiment, a plurality of spacers project from the tissue contacting surface and a plurality of the spacers each contains a suction orifice in fluid communication with the at least one suction channel.

In an exemplary embodiment, a plurality of tissue-penetrating temperature probes project laterally from the tissue contacting surface.

In an exemplary embodiment, the housing includes at least one irrigation orifice located proximate the fiber optic waveguide and at least one irrigation channel in fluid communication with the irrigation orifice.

In an exemplary embodiment, the reflector, as viewed sectionally along the axis of the side emitting diffuser, has a half-circular cross section about an axis parallel to the side emitting diffuser and the side emitting diffuser is located within the half-circle.

In an exemplary embodiment, the reflector, as viewed sectionally along the axis of the side emitting diffuser, defines a half-ellipse having a focus parallel to the side emitting diffuser and the side emitting diffuser is located proximate the focus.

In an exemplary embodiment, the side emitting diffuser, the reflector and the tissue contacting surface have a length in a range between five and ten centimeters.

In an exemplary embodiment, the side emitting diffuser terminates distally into a structure selected from the group including a corner cube retro-reflector, a right angle prism, and a multilayer dielectric mirror.

Also in accordance with the present invention, an exemplary embodiment of tissue ablation apparatus includes a fiber optic waveguide including a flexible side emitting diffuser having energy emission substantially uniform over its length; a plurality of tissue contacting surfaces operatively connected to the side emitting diffuser; tissue spacing means operatively connected to the side emitting diffuser for substantially fixing a lateral separation between the side emitting diffuser and a tissue which is to be illuminated with laser energy; means for temporarily anchoring the tissue contacting surfaces to a tissue which is to be illuminated with laser energy; and an elongate, flexible reflector operatively connected to the side emitting element and maintained at a predetermined lateral separation from the side emitting diffuser. The reflector extends substantially alongside the side emitting diffuser and partially surrounds the side emitting diffuser. The reflector and the side emitting diffuser define at least one convergent beam at a predetermined lateral distance from the side emitting diffuser. The predetermined lateral separation and the predetermined lateral distance are substantially unaffected by flexion of the side emitting diffuser and the reflector over a predetermined flexional range. The tissue contacting surfaces define an area of contact at a substantially fixed lateral distance from the side emitting diffuser. The convergent beam is defined at a predetermined location within a predetermined range of lateral distances from the side emitting diffuser, the range beginning at the area of contact and extending a predetermined distance beyond the area of contact.

Also in accordance with the present invention, a method for creating a lesion in a biological tissue includes the steps of providing a fiber optic waveguide including a flexible side emitting diffuser having energy emission substantially uniform over its length; positioning the side emitting diffuser over substantially its entire length to a desired portion of a biological tissue surface in which a lesion is to be created; fixing the side emitting diffuser at a predetermined distance from the desired portion of the biological tissue surface, the predetermined distance being substantially constant for all portions of the side emitting diffuser; and providing an elongate, flexible reflector in predetermined spatial relation to the side emitting diffuser. The reflector partially surrounds the side emitting diffuser. The reflector extends substantially the length of the side emitting diffuser, at a distance therefrom, the distance being substantially constant over the length thereof and substantially independent of flexion of the side emitting diffuser and the reflector. The reflector and the side emitting diffuser define at least one convergent beam at a predetermined distance from the side emitting diffuser, the convergent beam being defined at a predetermined location within a predetermined range of distances from the side emitting diffuser, the range extending a predetermined distance into the biological tissue. Also included is the step of providing laser energy to the fiber optic waveguide at a predetermined power level for a predetermined time period.

The spacing of the reflector and the side emitting fiber provide a convergent beam, and the spacing of the tissue contacting surfaces allow the beam to converge so that it is no closer than the distal wall of the tissue that is under treatment. This beam shape and placement will allow the energy density to remain high as the beam traverses the tissue, while possibly not exactly equal through the tissue thickness, at least compensating by greater concentration for the decrease in energy.

In an exemplary method, the step of approximating the side emitting diffuser over substantially its entire length to a desired portion of a biological tissue surface in which a lesion is to be created includes a step of bending the side emitting diffuser to conform to a curvature of the biological tissue, and the step of fixing the side emitting diffuser at a predetermined distance from the desired portion of the biological tissue surface includes a step of providing a plurality of tissue contacting surfaces, each operatively connected to the side emitting diffuser, each including a suction orifice, and the further step of providing suction to the suction orifices.

An exemplary method further includes the steps of providing a plurality of temperature probes projecting from the tissue contacting surfaces, placing the temperature probes in contact with the biological tissue, and, with the temperature probes, measuring a tissue temperature after beginning the step of providing laser energy to the fiber optic waveguide.

An exemplary method further includes the step of providing cooling fluid at a location proximate the side emitting diffuser.

### BRIEF DESCRIPTION OF THE DRAWINGS

For a further understanding of the objects and advantages of the present invention, reference should be had to the following detailed description, taken in conjunction with the accompanying drawings, in which like parts are given like reference numbers and wherein:

FIG. 1 is a bottom view of a tissue ablation apparatus in accordance with the present invention;

FIG. 2 is a side view of the apparatus of FIG. 1 detailed for a circular reflector

FIG. 3 is a top view of the apparatus of FIG. 1

FIG. 4 is a diagrammatic cross-sectional view of the apparatus of FIGS. 1, 2 and 3 in which the reflector is circular;

FIG. 5 a diagrammatic cross-sectional view of the apparatus of FIG. 1 in which the reflector is elliptical; and

FIG. 6 is a partial view showing the entry end of the cooling/irrigation elements.

### DETAILED DESCRIPTION

The present invention is an energy delivery system and method for performing laser ablation procedures using side emitting optical fibers emitting energy from a laser source to tissue to be treated. The system employs a catheter that includes a side emitting long period grating diffuser, in an exemplary version in the range of for example 5-10 cm, imprinted on the distal end of an optical fiber waveguide to make continuous photocoagulation lesions for effective treatments. The side emitting fiber optic high energy delivery platform uniformly emits optical energy over the length of the diffuser. The diffuser is housed in a flexible extended optical reflector channel to increase the energy delivery efficiency of the laser source. A distributed temperature sensor array, for monitoring the in-depth temperature gradient in the tissue during the procedure, is embedded in the diffuser housing, and extends along the length of the tissue under treatment. A series of openings connected to a suction line allows the instrument to be firmly attached to the tissue under treatment. An optional cooling/irrigation line with circulating coolant to cool the diffuser and/or to irrigate the tissue, for example to prevent blood coagulation at the surface of the myocardium. The exemplary embodiment disclosed below can accomplish all these functions.

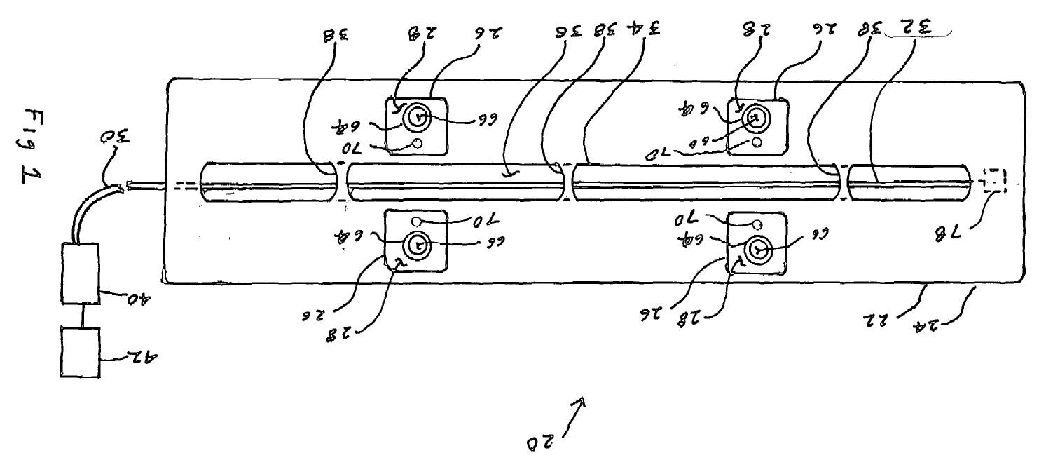

The invention will now be described with reference to FIGS. 1, 2, 3 and 4 showing an exemplary embodiment of tissue ablation apparatus in accordance with the present invention, shown generally at 20, including a catheter portion 22 comprising an elongate housing 24 having spacers 26 with tissue contacting surfaces 28 which define a catheter/tissue contact plane, and a fiber optic waveguide 30 operatively connected to side emitting diffuser 32 which is disposed on the housing 24 in an elongate open channel 34. An elongate reflector 36 is disposed on the surface of the elongate channel 34. The diffuser 32 extends through the channel 34 and is held in place by spaced apart reinforcing ribs or bridges 38. As will be seen the position of the diffuser 32 relative to the reflector 36 is important and it is determined by its location in passing through the reinforcing ribs 38. The preferred light source is a laser source 40. Operation of the apparatus is controlled by a control system 42. The distance between the reinforcing ribs is selected to ensure the most consistent placement of the side emitting diffuser 28 to the reflector with due regard for the amount of bending anticipated. The diffuser 32 extends slidably through perforations in the bridges 38.

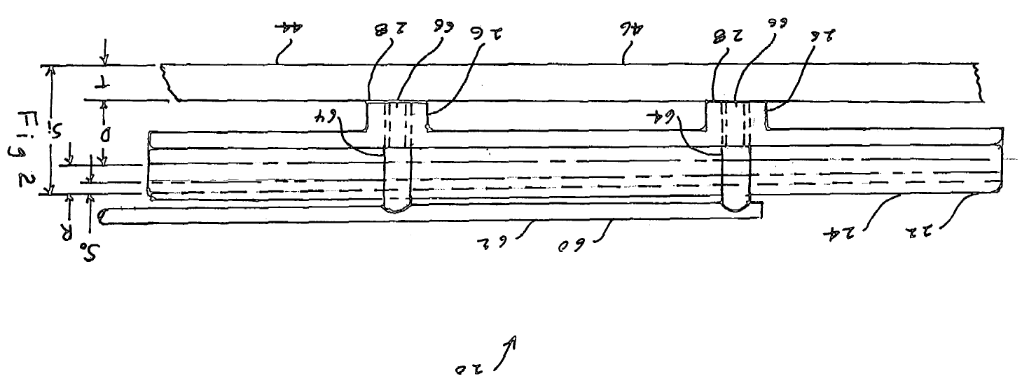

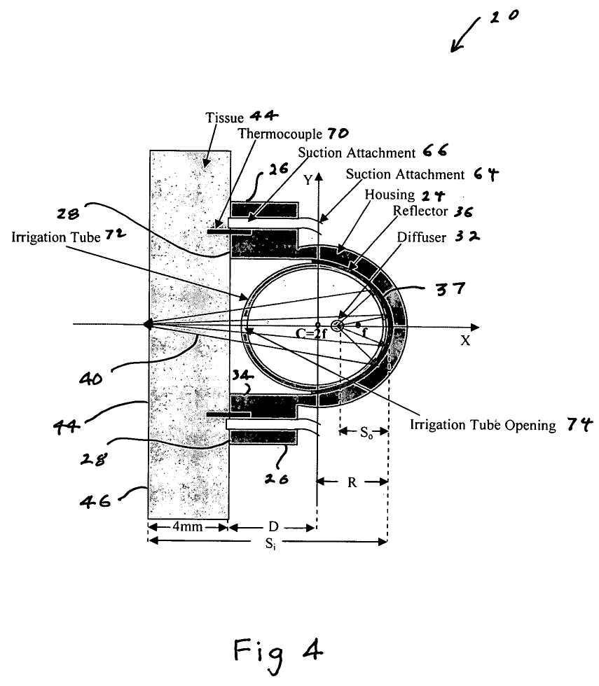

FIG. 2 and FIG. 4 show a preferred configuration of the elongate channel 34 and reflector 36, in this case, circular. When configured and fitted in proper spatial relationship as described below, the reflector 36 and the diffuser 32 define a convergent beam 40 of emitted laser light extending at a predetermined relationship to a tissue portion 44 under treatment. The reflector 36 is provided on the surface 37 of the channel 34 and reinforcing ribs 38 hold the diffuser 32 at a predetermined constant distance from the reflector 30 over substantially its entire length in the channel 34. The reinforcing ribs 38 also help keep the channel 34, and consequently the reflector 38 in proper shape.

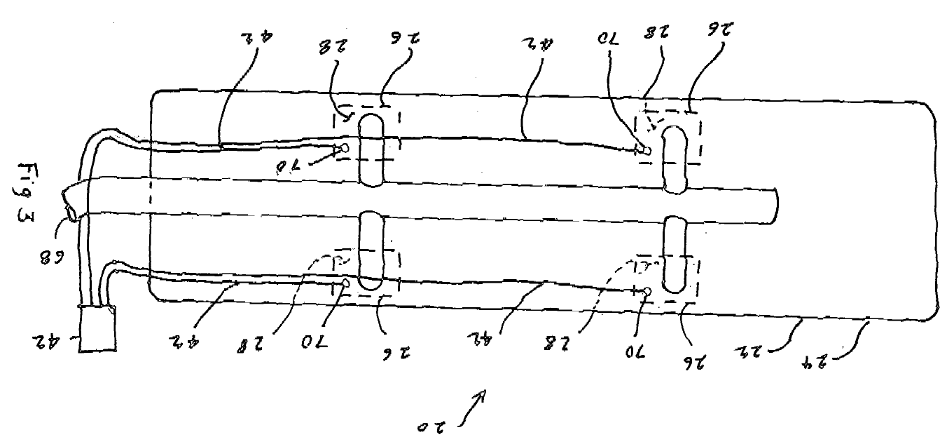

With continued reference to FIG. 1, FIG. 2, FIG. 3 and FIG. 4, in an exemplary embodiment, the housing 24 is formed by molding from Dow Corning 3120 RTV Silicone Rubber mixed with Dow Corning 1 Catalyst in which the housing 24 is approximately 11 centimeters in length, approximately two centimeters in width, and somewhat less than one centimeter laterally as measured from the tissue contacting surface 28 through the portion of the housing 24 that forms the channel 34.

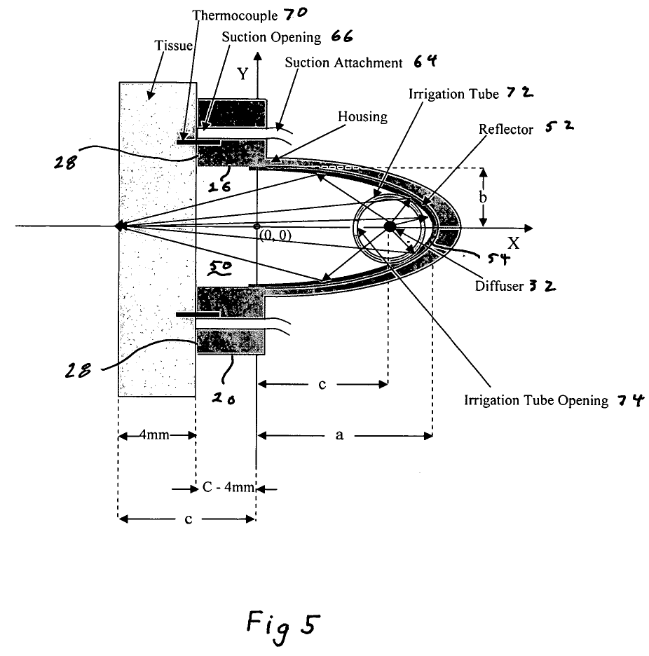

With reference to FIG. 1, FIG. 2, FIG. 3, FIG. 4 and FIG. 5 (FIG. 5 is described below) the preferred embodiment of the reflector 36 includes a film of gold leaf, five microns in thickness.

Referring to FIG. 4, the general relationships can be determined; in the case of the circular cross-section reflector 36. As the position (Sₒ) of the side emitting diffuser 32 moves between the focal point (f) and the center of the reflector 32 (C=2f), the diffuser image or focal point (Sᵢ) will move in the region outside the C=2f. That means Sᵢ≧2f. The tissue thickness is designated (T). For the reflector 32 the following obtains:

| | | | |
|---|---|---|---|
| 1 | 1 | 1 | 2 |
| Sₒ | Sᵢ | f | R |

and
Sᵢ = 2f + D + T;

where D is the distance from the center C of the reflector circle, and T is the thickness of the tissue under treatment, Sᵢ is the distance from the distal wall of the tissue under treatment, Sₒ is the selected distance of the diffuser 32 along the X axis to the reflector surface, R is the radius of the circle defined by the reflector surface.

In the exemplary application for treatment for atrial fibrillation, assuming the atrial tissue to have a thickness of about 4 mm:

Sᵢ=2f+D+4 mm.

The beam shape can be varied by selecting the desired point of placement of the side emitting diffuser between C an f to have the focal point of the beam at the selected place relative to the tissue under treatment; that selected place being preferable not closer than the distal wall of the tissue.

With reference to FIG. 1, FIG. 2 and FIG. 4 in the exemplary embodiment, the reflector 36 is oriented longitudinally in the channel 34 which has a circular cross section radius, R, about five to six millimeters. The reinforcing ribs 38 extend across the channel 34 and center the side emitting diffuser 28 within the curvature of the reflector 36 at a distance, Sᵢ, along the X axis (shown in FIG. 4) of about three millimeters from the reflector 36 surface, a location between the reflector 36 surface and the center of curvature, C. As can be seen in FIG. 4, reflected rays from the side emitting diffuser 32 converge at a location approximately four millimeters beyond the center of curvature of the reflector 36. This puts the point of convergence (also called the focal point) at about the distal wall 46 of the tissue 44. This will cause a more uniform energy distribution through the tissue thickness (as compared with a collimated beam), and along the tissue length, since more energy focus as the beam extends into the tissue depth will compensate for tissue energy absorption.

With reference to FIG. 5, in another alternative embodiment, the channel 50 has an elliptical cross section and the reflector 52 is oriented longitudinally on the channel surface 54. In the exemplary embodiment, the elliptical cross section has a width of six millimeters, a depth of 6.325 millimeters and one of its foci located 0.65 millimeter from the reflector 52. Reinforcing ribs 38 extend from the housing 24 and center the side emitting diffuser 32 within the curvature of the reflector 52 at a distance 0.65 millimeter along the X axis from the reflector 52, a location substantially coinciding with the other focus of the elliptical curvature of the reflector 52. As can be seen in FIG. 5, for the exemplary embodiment, reflected rays, forming a convergent beam, from the side emitting diffuser 32 converge at a location, a focal point, approximately four millimeters beyond the tissue contact surface 28, corresponding to a second focus of the elliptical curvature of the reflector 52 and at the distal wall of the exemplary 4 mm thick tissue. In such an exemplary embodiment, b=3 mm, c=6 mm, and a=6.325 mm. From conventional ellipse geometry,

b=a²−c².

With continued reference to FIG. 1, FIG. 2, FIG. 3, FIG. 4 and FIG. 5 a suction system is created through the spacers 26 by tubing 60 having a central tube 62 and branches 64 that “tee” off the central tube 62 through the spacers 26 to establish suction orifices 66 at the tissue contacting surfaces 28 of each spacer 26. The end 68 of the suction tubing 60 can be attached to a suction apparatus that is in turn controlled by the control system 42

The apparatus preferably has a means for monitoring the temperature along the length of the tissue under treatment, and preferably the temperature gradient through the tissue thickness. In an exemplary embodiment of such a means, with continued reference to FIG. 1, FIG. 3, FIG. 4, and FIG. 5 a temperature probe 70 extends through and beyond the spacers 26 into the tissue to a selected depth, each probe 70 being connected to a temperature lead 42. An exemplary temperature probe 70 is a Quick Disconnect J Type, Iron-Constantin Thermocouple (with SS sheath, 0.020 inch outside diameter, grounded junction) embedded in the spacers 26 and projecting from the tissue contacting surfaces 28 of the spacers 26. A greater number of probes provides a more detailed temperature measuring capability, but at the disadvantage of greater mass and complexity and reduced flexibility. These disadvantages can be mitigated by reducing the size of the temperature probes 70.

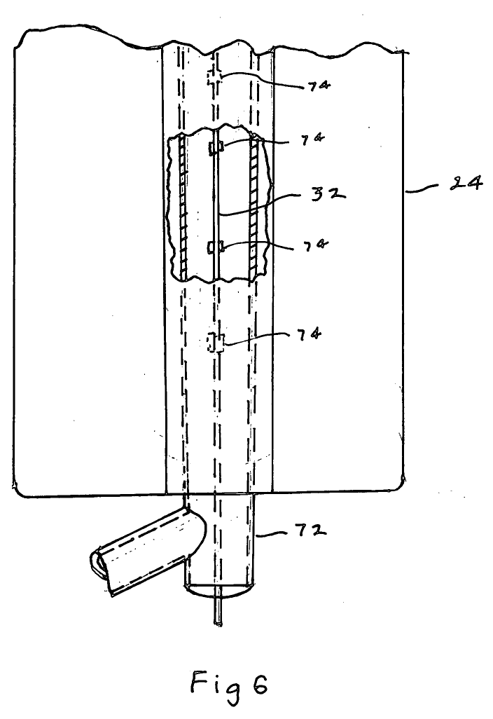

With reference to FIGS. 4, 5 and 6, in an optional embodiment, the catheter 24 includes an irrigation tube 72 that extends the length of the channel 34 and 50 respectively and has a series of spaced apart irrigation openings 74 located proximate the tissue engaging plane so as to direct irrigation fluid to the tissue under treatment. In this embodiment, the side emitting diffuser 32 is inside the irrigation tube 72. However, an irrigation tube can be located at any other convenient, effective location proximate the catheter 24 so as to irrigate the tissue under treatment. Saline solution may be delivered through the irrigation opening 74 with sufficient pressure to flush debris away from the side emitting diffuser 32 and in addition can function to cool the side emitting diffuser 32, or to cool tissues during use of the apparatus.

In the present exemplary embodiment, the side emitting diffuser 32 is characterized by constant longitudinal radiant emission, i.e., the radiant emission is substantially constant over the entire length of the side emitting diffuser 32. Laser light energy supplied via the fiber optic waveguide 30 travels longitudinally into the side emitting diffuser 32. As the energy is transmitted within the side emitting diffuser 32, a portion of the energy is scattered and escapes laterally. The remaining energy, somewhat diminished, is transmitted longitudinally. Because the transmitted power density decreases with increasing distance along the side emitting diffuser 28, a correspondingly increasing portion of the energy must be scattered to hold the emitted (escaping) power density constant with increasing distance along the side emitting diffuser 28. Thus, the optical properties of the fiber optic waveguide 26 must change over the length of the side emitting diffuser 38 to provide constant power emission. Constant power distribution means are disclosed in U.S. Pat. Nos. 6,205,263 and 7,006,718.

With reference to FIG. 1, FIG. 2, FIG. 4 and FIG. 5, the reflector 36 (in FIG. 4) and 52 (in FIG. 5) partially surrounds the side emitting diffuser 32 and is held at a predetermined spacing from the side emitting diffuser 32, by perforated ribs 38, the lateral separation being substantially constant over the length of the side emitting diffuser 28, as explained in greater detail above, in order to establish the desired beam configuration.

With reference to FIG. 1, FIG. 2, FIG. 4 and FIG. 5 the tissue contacting surfaces 28 define a substantially planar area of contact at a substantially fixed lateral distance from the side emitting diffuser 32, as explained in detail above. This planar area is defined as the tissue/catheter contact plane. As seen best in FIG. 2, FIG. 4 and FIG. 5, this planar area of contact corresponds to the surfaces 28 of the spacers 26. The convergent beam 32 is defined at a predetermined location within a predetermined range of lateral distances from the side emitting diffuser 28, the range beginning at the tissue/catheter contact plane and extending a predetermined distance beyond the tissue/catheter contact plane. In the preferred embodiment the convergent beam 32 ends at a focal point not closer that the distal wall 46 of the tissue 44 under treatment

In the exemplary embodiment, the side emitting diffuser 32, the reflectors 36 and 52, and the tissue contacting surfaces 28 have a length in a range between five and ten centimeters.

The housing 24, is deformable between a straight configuration and a curved configuration in order to be placed in contact with or to assume, when suction is applied, the curvature of the tissue under treatment such as the atrial wall, and a substantially fixed predetermined separation is maintained between the side emitting diffuser 32 and the reflectors 36 and 52, and between the reflectors 36 and 52 and the tissue contacting surfaces 28 at the straight configuration, at the curved configuration, and at intermediate configurations.

In one exemplary version of this embodiment, the side emitting diffuser 32 includes a matted wall diffuser formed by removing the fiber cladding and roughening the surface of the exposed core of a 200 micrometer or 400 micrometer fiber with diamond sandpaper or with another burnishing tool until sufficient scattering is obtained.

In the herein described exemplary embodiment, the side emitting diffuser 32 includes a long period grating and the side emitting diffuser 32 emits energy substantially uniformly over its length. A preferable range of the supplied laser energy is at wavelength between 970 and 1060 nanometers, more preferably between 970 and 980 nanometers. An exemplary side emitting diffuser 32 with a long period grating is produced utilizing a germanium-doped fiber with a 200 micrometer core diameter, a 20 micrometer cladding, a numerical aperture of 0.37 and a polyamide buffer. Another exemplary side emitting diffuser 28 with a long period grating is produced utilizing a germanium-doped fiber with a 400 micrometer core diameter, a 40 micrometer cladding, a numerical aperture of 0.37 and a Tefzel buffer. In both of these, the fibers (obtained from Ceramoptech GmbH, Siemens str. 44, 52121, Bonn, Germany) are hydrogen loaded. The buffer is removed, chemically or mechanically, for a length of one centimeter greater than the intended length of the side emitting diffuser 32. A periodic scattering structure is written into the fiber using 10-nanosecond pulses from a KrF excimer laser emitting at 248 nanometers. The fiber is irradiated through an amplitude mask the radiant exposure on the fiber during the pulse being as high as 8.5 Joule per square centimeter.

In these exemplary embodiments, laser power is provided by coupling the fiber optic waveguide 26 to a 25 watt continuous wave laser diode (Apollo Instruments, Irvine Calif.) emitting at a wavelength of 976 nanometers.

With reference to FIG. 1 in an exemplary embodiment, the side emitting diffuser 32 terminates distally into a retro-reflective structure 78 which may be a corner cube retro-reflector a right angle prism, or a multilayer dielectric mirror, with the result that a significant portion of the residual transmitted energy which might otherwise overheat the fiber is instead returned to the side emitting diffuser 32, thereby rendering the apparatus more efficient and reducing the likelihood of overheating of the distal end of the side emitting diffuser 32 or of nearby tissues or fluids.

The reflector and the side emitting diffuser define a convergent beam extending to a predetermined lateral distance from the side emitting diffuser 32. This distance, usually approximately 0.5 centimeter, may be varied by altering the curvature of the reflector or the position of the side emitting diffuser relative to the reflector. Preferably, the convergent beam focal point (or image point) should occur at or about a depth of four millimeters into the tissue (referring to atrial wall tissue), that, generally, is at or slightly beyond the distal wall of the tissue. The spacers 26 establish the distance between the reflector and the tissue when the apparatus is positioned on the tissue. Thus, the convergence should occur approximately four millimeters beyond the reach of the spacers 265, which define a substantially planar area of contact between the apparatus and the tissue. With spacers 26 projecting two millimeters from the tissue contacting surface 28, the convergence is therefore desired at approximately six millimeters from the tissue contacting surface 28.

Also in this preferred embodiment, the housing 24 is flexible enough to tolerate a range of flexion without buckling. Within this range of flexion, the reflector and the side emitting diffuser will remain at substantially constant separation at different degrees of flexion, even though their respective radii of curvature are different, because the reinforcing ribs 38 provide only lateral restraint for the side emitting diffuser 28 of the fiber optic wave guide, but not longitudinal restraint. The side emitting diffuser 28 is free to slide longitudinally relative to each reinforcing rib 34.

As discussed above the reflector cross section may have the form either of an ellipse or of a circle. With either of these curvatures, the overriding objective is to concentrate reflected radiation within a narrow strip of tissue, approximately a few, up to five, millimeters wide, at depths approaching four millimeters into the tissue, in a manner tending to offset the absorption and scattering of radiation at the surface. Reflected rays enter the tissue surface over a strip nearly the width of the reflector, but at varying angles such that they tend to converge at a point corresponding to the image the reflector forms of the side emitting diffuser, this image occurring several centimeters beneath the tissue surface. Thus, the supplied power may be adjusted so that the combined power density of the direct and reflected radiation incident at nearly normal angles at those locations on the tissue surface closest to the side emitting diffuser is below the level that is expected to char or vaporize the tissue at those locations, yet sufficient to create the desired permanent lesion. At greater depths in the tissue, where absorption and scattering by the intervening tissue have reduced the power density of the nearly normally incident radiation to a sub-therapeutic level, the convergence of reflected radiation entering at lower angles and lower density boosts the total power density at these greater depths so that a permanent lesion is created at these depths.

Also in this preferred embodiment, it is preferable to create lesions between five and ten centimeters long with a single application. Thus, the side emitting diffuser and reflector have lengths in a range between five and ten centimeters.

Also in accordance with the present invention, a method is provided for creating a lesion in a biological tissue utilizing the above-described preferred embodiment of tissue ablation apparatus. It will be appreciated that successful treatment of AF with this apparatus calls for quickly and efficiently creating a continuous, elongated lesion of precisely controlled placement, depth and severity on curved, living, moving heart tissue. Constant emission over the length of the side emitting diffuser provides an ability simultaneously to irradiate a strip of tissue up to ten centimeters long. The reflector provides an ability to deliver an increased portion of the emitted energy, which escapes the fiber at all azimuthal angles, to the tissue so that the total power delivered through the optical fiber waveguide may be reduced to levels the side emitting diffuser may more easily tolerate. Additionally, the reflector, with appropriate curvature and separation from the side emitting diffuser concentrates light at a predetermined distance from the tissue contacting plane, making it possible to create a lesion at depth without over-irradiating the tissue surface.

When the catheter is applied to the heart and suction is provided to the suction orifices 56 on the spacers 26 via the suction tube system 60 the tissue contacting surfaces 28 of the spacers 26 are temporarily anchored to the tissue, fixing the actual separation at a value such that the convergent beam will occur within a desired range of depths in the tissue. The beam is shaped so that its focal point is no closer than the distal wall of the tissue under treatment which provides compensation for attenuation of power with depth by concentrating the power. It is permissible that the focal point be slightly beyond the distal wall, but it is considered that allowing the focal point to be inside the tissue will be counter to the goal of keeping the power with depth as constant as possible. Being flexible, the housing, reflector and side emitting diffuser conform to the curvature of the heart tissue while maintaining the predetermined separation between the side emitting element and the reflector. Thus, the apparatus is temporarily fixed on the moving heart in a position affording an opportunity for successful treatment.

As the side emitting diffuser is able to slide in the perforated bridges, and although it may be fixed at one end of the housing, this sliding allows it to maintain a closely consistently curved curvature and therefore maintain a closely equal distance from the reflector along the X axis. The reflector itself is less consistent when curved and to be as closely equal in curvature to the side emitting diffuser, it should be as thin as practical in the portion defining the channel. However as can be appreciated from the foregoing description, there is some acceptable variation in the spacing of the side emitting diffuser to the reflector, such as seen in FIG. 4 where the side emitting diffuser can move within the range between C and f, and the variation in that movement can be controlled by the design dimensions to still provide the correct beam shape and focal point. The same is true for the elliptical shaped reflector of FIG. 5 as will be apparent to those skilled in the art.

Although the foregoing embodiments are described in the context of using laser energy in the visible light portion of the spectrum, it is apparent to those skilled in the art that the energy can be provided by sources in other portions of the electromagnetic spectrum that would result in the requisite side emission and beam shape and required energy delivered to the tissue to cause lesions by ablation. Such alternative sources even in the light portion of the spectrum need not be laser if sufficient power can be delivered to the fiber optic waveguide by the light source.

As discussed hereinabove, laser energy of appropriate wavelength is delivered to the side emitting diffuser at a predetermined power level for a predetermined time period. Saline irrigation fluid may be delivered as needed to clear debris from the space intervening between the apparatus and the tissue and also to cool the side emitting diffuser and the tissue.

In pursuit of safety and efficacy in performing the procedure, as well as in pursuit of data for validating and optimizing the procedure, temperature data are acquired via the tissue-penetrating temperature probes 70 that project from the spacers 26. Tissue temperature at various depths and at various distances from the lesion site may be observed as radiation is delivered. Power may be interrupted, or cooling initiated, if an observed temperature exceeds a limit previously associated with an unacceptable risk. Temperature data may later be correlated with postoperative outcomes and utilized to modify the procedure or the apparatus.

While the invention is described in terms of a specific embodiment, other embodiments could readily be adapted by one skilled in the art. Accordingly, the scope of the invention is limited only by the following claims.

The foregoing Detailed Description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. This disclosure has been made with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “step(s) for . . . ”

1. Tissue ablation catheter comprising;
an elongate housing having one or more tissue contacting surfaces defining a catheter/tissue contact plane;
an elongate reflector disposed on said housing at a predetermined spatial relationship to said catheter/tissue contact plane;
a fiber optic side emitting diffuser, said side emitting diffuser being disposed on said housing in predetermined spatial relationship to said reflector to provide an elongate convergent beam having a focal point beyond said catheter/tissue contact plane;
a source of electromagnetic energy connected to the side emitting diffuser for providing emission of energy from the side emitting diffuser;
whereby tissue in contact with the catheter at the catheter/tissue contact plane will have the elongate convergent beam extending into the tissue to cause ablation of the tissue along at least a portion of the length of the elongate beam.

2. Tissue ablation catheter of claim 1 wherein the energy is laser energy.

3. Tissue ablation catheter of claim 1, wherein said one or more tissue contacting surfaces include a plurality of spaced apart suction orifices in communication with a suction system said orifices being adapted to contact tissue to maintain it in a predetermined spatial relationship to said reflector defined by said catheter/tissue contact plane.

4. Tissue ablation catheter of claim 1, wherein said one or more tissue contacting surfaces includes a plurality of temperature probes extending away from said catheter;
whereby said temperature probes will extend into tissue under treatment to allow temperature monitoring.

5. Tissue ablation catheter of claim 1, wherein said housing includes at least one irrigation orifice located proximate said side emitting diffuser and at least one irrigation channel in fluid communication with said irrigation orifice to allow irrigation fluid to be directed at tissue under treatment.

6. Tissue ablation catheter of claim 1, wherein one or more tissue contacting surfaces comprise a plurality of tissue contacting spacers arranged along the length of the housing.

7. Tissue ablation catheter of claim 1, wherein said side emitting diffuser emits energy substantially uniformly over its length.

8. Tissue ablation catheter of claim 1, wherein said side emitting diffuser's spatial relationship to said reflector is substantially constant over the length of said reflector.

9. Tissue ablation catheter of claim 1, wherein said side emitting diffuser's spatial relationship to said reflector is selected from a range of distances so as to provide a convergent beam configured to have a focal point at a predetermined location within a predetermined range of lateral distances from said catheter/tissue contact plane.

10. Tissue ablation catheter of claim 1, wherein said side emitting diffuser, and said reflector, have a length in a range between five and ten centimeters.

11. Tissue ablation catheter of claim 1, wherein said housing, said one or more tissue contacting surfaces, said side emitting diffuser and said reflector are deformable between a straight configuration and a curved configuration;
whereby the catheter may conform to the curvature of tissue under treatment.

12. Tissue ablation catheter of claim 1, wherein said side emitting diffuser includes a long period grating.

13. Tissue ablation catheter of claim 1, wherein said side emitting diffuser terminates distally into a structure selected from the group including; a polished diffuser tip coated with a reflective mirror; a metallic mirror with a heat sink; a Bragg reflector; a diffuser tip beveled in the shape of a wedge or cone; a dielectric film deposited on the tip of the reflector; a corner cube retro-reflector, a right angle prism made from a material with a refractive index different from that of the diffuser, and a multilayer dielectric mirror;
said structure being effective to retro-reflect radiation at the distal end of the side emitting diffuser.

14. Tissue ablation catheter of claim 1 comprising a plurality of tissue contacting surfaces spaced apart along the length of said elongate housing.

15. Tissue ablation catheter of claim one wherein said reflector and said side emitting diffuser are placed in a channel in said housing, said channel having an opening toward said tissue/catheter contact plane.

16. Tissue ablation catheter of claim 2 wherein said laser energy is supplied at a wavelength between about 970 and about 1060 nanometers.

17. Tissue ablation catheter of claim 15 wherein said channel has perforated spaced apart bridges along its length and said side emitting diffuser passes slidably through a perforation in each of said bridges said perforation being placed at a predetermined space from said reflector to locate said side emitting diffuser in said predetermined spatial relationship to said reflector.

18. Tissue ablation catheter of claim 16 wherein said wavelength is from about 970 to about 980 nanometers

19. Tissue ablation catheter, comprising;
an elongate, flexible housing having one or more tissue contacting surfaces defining a tissue/catheter contact plane;
a plurality of suction orifices formed in portions of said housing and being open at said tissue/catheter plane;
a suction providing system in fluid communication with said suction orifices;
a fiber optic side emitting diffuser having energy emission substantially uniform over its length, said side emitting diffuser being disposed within said housing at a predetermined lateral separation from said tissue/catheter contact plane; and
an elongate, flexible reflector disposed within said housing at predetermined lateral separation from said side emitting diffuser, said reflector extending substantially alongside said side emitting diffuser and partially surrounding said side emitting diffuser;
said reflector and said side emitting diffuser defining an elongate convergent beam at a predetermined lateral distance from said side emitting diffuser;
said predetermined lateral separation and said predetermined lateral distance being substantially unaffected by flexion of said side emitting diffuser, and said reflector over a predetermined flexional range;
said tissue contacting surface defining a tissue/catheter contact plane at a substantially fixed lateral distance from said side emitting diffuser,
said convergent beam being defined as having its focal point at a predetermined location within a predetermined range of lateral distances from said side emitting diffuser beyond said tissue/catheter contact plane.

20. Tissue ablation catheter of claim 19, wherein said convergent beam focal point is defined at a location with relation to tissue under treatment to be not closer than the distal wall of said tissue.

21. Tissue ablation catheter of claim 19, wherein a plurality of spacers project from said housing having surfaces that define said tissue/catheter contact plane and a plurality of said spacers each contains a suction orifice in communication with said suction system.

22. Tissue ablation catheter of claim 19, wherein a plurality of tissue-penetrating temperature probes project laterally from housing a selected distance sufficient beyond said tissue/catheter contact plane to allow monitoring of the temperature in tissue under treatment.

23. Tissue ablation catheter of claim 19, wherein said housing includes at least one irrigation orifice located proximate said side emitting diffuser and at least one irrigation channel in fluid communication with said irrigation orifice.

24. Tissue ablation catheter of claim 19 wherein said reflector, as viewed sectionally along the axis of said side emitting diffuser, has a cross section defining a segment of a circle about an axis parallel to said side emitting diffuser and said side emitting diffuser is located between the center of the circle and the focus, f, of the circle.

25. Tissue ablation catheter of claim 19, wherein said reflector, as viewed sectionally along the axis of said side emitting diffuser, defines an ellipse having a focus parallel to said side emitting diffuser and said side emitting diffuser is located proximate said focus.

26. Tissue ablation catheter of claim 19, wherein said side emitting diffuser, said reflector a have a length in a range between five and ten centimeters.

27. Tissue ablation catheter of claim 19, wherein said side emitting diffuser terminates distally into a structure for providing retro-reflection.

28. Tissue ablation apparatus, comprising;
a flexible side emitting diffuser having energy emission substantially uniformly over its length;
a tissue spacing means defining a tissue/catheter contact plane said spacing means being operatively connected to said side emitting diffuser for substantially fixing a lateral separation between said side emitting diffuser and a tissue in contact with said tissue spacing means which is to be illuminated with laser energy;
means for temporarily anchoring said tissue contacting surfaces to a tissue which is to be illuminated with laser energy; and
an elongate, flexible reflector operatively connected to said side emitting diffuser and maintained at a predetermined lateral separation from said side emitting diffuser, said reflector extending substantially alongside said side emitting diffuser and partially surrounding said side emitting diffuser,
said reflector and said side emitting diffuser defining at least one convergent beam extending to a focal point at a predetermined lateral distance from said reflector,
said predetermined lateral separation and said predetermined lateral distance being substantially unaffected by flexion of said side emitting diffuser and said reflector over a predetermined flexional range,
said tissue/catheter contact plane defining an area of contact at a substantially fixed lateral distance from said reflector,
said convergent beam being defined at a predetermined location within a predetermined range of lateral distances from said reflector, said range beginning at said tissue/catheter contact plane and extending a predetermined distance beyond it.

29. A method for creating a lesion in a biological tissue, the method including the steps of:
providing a flexible side emitting diffuser having energy emission substantially uniformly over its length;
fixing said side emitting diffuser at a predetermined distance from the desired portion of the biological tissue surface, said predetermined distance being substantially constant along the length of said side emitting diffuser
providing an elongate, flexible reflector in predetermined spatial relation to said side emitting diffuser,
said reflector partially surrounding said side emitting diffuser, said reflector extending substantially the length of said side emitting diffuser, at a distance therefrom, said distance being substantially constant over the length thereof and substantially independent of flexion of said side emitting diffuser and said reflector,
said reflector and said side emitting diffuser defining at least one convergent beam at a predetermined distance from said side emitting diffuser when electromagnetic energy is passed through said side emitting diffuser,
said convergent beam being defined at a predetermined location within a predetermined range of distances from said side emitting diffuser, said range extending a predetermined distance into the biological tissue; and
providing electromagnetic energy to said side emitting diffuser at a predetermined power level for a predetermined time period.

30. The method set forth in claim 29 wherein said step of fixing said side emitting diffuser at a predetermined distance from the desired portion of the biological tissue surface includes a step of providing a plurality of tissue contacting surfaces, each operatively connected to said side emitting diffuser, each including a suction orifice, and the further step of providing suction to said suction orifices.

31. The method set forth in claim 30, further including the steps of:
providing a plurality of temperature probes projecting from said tissue contacting surfaces,
placing said temperature probes in contact with the biological tissue, and
with said temperature probes, monitoring the temperature of the tissue during said predetermined time period.

32. The method set forth in claim 30, further including the step of providing cooling fluid at a location proximate said side emitting diffuser.

33. A method for treating atrial fibrillation, the method including the steps of:
providing a flexible side emitting diffuser having energy emission substantially uniformly over its length;
fixing said side emitting diffuser at a predetermined distance from the desired portion of the heart surface, said predetermined distance being substantially constant for all portions of said side emitting diffuser;
providing an elongate, flexible reflector in predetermined spatial relation to said side emitting diffuser,
said reflector partially surrounding said side emitting diffuser, said reflector extending substantially the length of said side emitting diffuser, at a distance therefrom, said distance being substantially constant over the length thereof and substantially independent of flexion of said side emitting diffuser and said reflector,
said reflector and said side emitting diffuser defining at least one elongate convergent beam at a predetermined distance from said side emitting diffuser,
said convergent beam being defined at a predetermined location within a predetermined range of distances from said side emitting diffuser, said range extending a predetermined distance into the heart tissue; and
providing laser energy to said fiber optic waveguide at a predetermined power level until at least one elongated continuous lesion of predetermined depth and severity is created in the heart tissue.

34. The method set forth in claim 33 wherein said step of fixing said side emitting diffuser at a predetermined distance from the desired portion of the heart surface includes a step of providing a plurality of tissue contacting surfaces, each operatively connected to said side emitting diffuser, each including a suction orifice, and the further step of providing suction to said suction orifices.

35. The method set forth in claim 33 further including the steps of:
providing a plurality of temperature probes projecting from said tissue contacting surfaces,
placing said temperature probes in contact with the heart tissue, and
with said temperature probes, monitoring the temperature during the procedure.

36. The method set forth in claim 33, further including the step of providing cooling fluid at a location proximate said side emitting diffuser.
